SEQUENCE LISTING (36 sequences)

SEQ ID NO: 1 - DNA, 100 bp, Homo sapiens
gtcctgaggccagcctaagtcgcttcaaaatggaacgaaggcgtttgtggggttccattcagagccgatacatcagcatgagtgtgtggacaagcccacg

SEQ ID NO: 2 - DNA, 100 bp, Homo sapiens
caatgagcggttccgctgccctgaggcactcttccagccttccttcctgggcatggagtcctgtggcatccacgaaactaccttcaactccatcatgaag

SEQ ID NO: 3 - DNA, 24 bp, Artificial Sequence
  Synthetic
cctaagtcgcttcaaaatggaacg

SEQ ID NO: 4 - DNA, 21 bp, Artificial Sequence
  Synthetic
aggccagcctaagtcgcttca

SEQ ID NO: 5 - DNA, 23 bp, Artificial Sequence
  Synthetic
gtcgcttcaaaatggaacgaagg

SEQ ID NO: 6 - DNA, 24 bp, Artificial Sequence
  Synthetic
cagcctaagtcgcttcaaaatgga

SEQ ID NO: 7 - DNA, 25 bp, Artificial Sequence
  Synthetic
tccacacactcatgctgatgtatcg

SEQ ID NO: 8 - DNA, 25 bp, Artificial Sequence
  Synthetic
cactcatgctgatgtatcggctctg

SEQ ID NO: 9 - DNA, 24 bp, Artificial Sequence
  Synthetic
tcatgctgatgtatcggctctgaa

SEQ ID NO: 10 - DNA, 25 bp, Artificial Sequence
  Synthetic
acactcatgctgatgtatcggctct

SEQ ID NO: 11 - DNA, 26 bp, Artificial Sequence
  Synthetic
aaggcgtttgtggggttccattcaga

SEQ ID NO: 12 - DNA, 26 bp, Artificial Sequence
  Synthetic
acgaaggcgtttgtggggttccattc

SEQ ID NO: 13 - DNA, 12 bp, Artificial Sequence
  Synthetic
ttgtggggttcc

SEQ ID NO: 14 - DNA, 26 bp, Artificial Sequence
  Synthetic
acgaaggcgtttgtggggttccattc

SEQ ID NO: 15 - DNA, 14 bp, Artificial Sequence
  Synthetic
cgtttgtggggttc

SEQ ID NO: 16 - DNA, 13 bp, Artificial Sequence
  Synthetic
tttgtggggttcc

SEQ ID NO: 17 - DNA, 21 bp, Artificial Sequence
  Synthetic
tgccctgaggcactcttccag

SEQ ID NO: 18 - DNA, 20 bp, Artificial Sequence
  Synthetic
tgccctgaggcactcttcca

SEQ ID NO: 19 - DNA, 19 bp, Artificial Sequence
  Synthetic
tgccctgaggcactcttcc

SEQ ID NO: 20 - DNA, 20 bp, Artificial Sequence
  Synthetic
gccctgaggcactcttccag

SEQ ID NO: 21 - DNA, 19 bp, Artificial Sequence
  Synthetic
ccctgaggcactcttccag

SEQ ID NO: 22 - DNA, 19 bp, Artificial Sequence
  Synthetic
tgccctgaggcactcttcc

SEQ ID NO: 23 - DNA, 19 bp, Artificial Sequence
  Synthetic
ccctgaggcactcttccag

SEQ ID NO: 24 - DNA, 26 bp, Artificial Sequence
  Synthetic
tggagttgaaggtagtttcgtggatg

SEQ ID NO: 25 - DNA, 24 bp, Artificial Sequence
  Synthetic
gagttgaaggtagtttcgtggatg

SEQ ID NO: 26 - DNA, 23 bp, Artificial Sequence
  Synthetic
tggagttgaaggtagtttcgtgg

SEQ ID NO: 27 - DNA, 23 bp, Artificial Sequence
  Synthetic
ggagttgaaggtagtttcgtgga

SEQ ID NO: 28 - DNA, 25 bp, Artificial Sequence
  Synthetic
ccttccttcctgggcatggagtcct

SEQ ID NO: 29 - DNA, 23 bp, Artificial Sequence
  Synthetic
ccttccttcctgggcatggagtc

SEQ ID NO: 30 - DNA, 22 bp, Artificial Sequence
  Synthetic
cttccttcctgggcatggagtc

SEQ ID NO: 31 - DNA, 25 bp, Artificial Sequence
  Synthetic
ccttccttcctgggcatggagtcct

SEQ ID NO: 32 - DNA, 21 bp, Artificial Sequence
  Synthetic
gccctgaggcactcttccagc

SEQ ID NO: 33 - DNA, 23 bp, Artificial Sequence
  Synthetic
tgaaggtagtttcgtggatgcca

SEQ ID NO: 34 - DNA, 18 bp, Artificial Sequence
  Synthetic
cgutugugggguuccauu

SEQ ID NO: 35 - DNA, 22 bp, Artificial Sequence
  Synthetic
cacactcatgctgatgtatcgg

SEQ ID NO: 36 - DNA, 21 bp, Artificial Sequence
  Synthetic
aagtcgcttcaaaatggaacg

(19) United States
(12) Patent Application Publication
(10) Pub. No.: US 2014/0072971 A1
(43) Pub. Date: Mar. 13, 2014

(54) PRAME DETECTION ASSAYS

(71) Applicants: Jeffrey D. Wuitschick, Wauwatosa, WI (US); Shihai Huang, Lincolnshire, IL (US)

(72) Inventors: Jeffrey D. Wuitschick, Wauwatosa, WI (US); Shihai Huang, Lincolnshire, IL (US)

(73) Assignee: ABBOTT LABORATORIES, Abbott Park, IL (US)

(21) Appl. No.: 14/015,251

(22) Filed: Aug. 30, 2013

Related U.S. Application Data
(60) Provisional application No. 61/695,180, filed on Aug. 30, 2012.

(51) Int. Cl. C12Q 1/68

(52) U.S. Cl. CPC C12Q 1/6886
USPC 435/6.11; 536/24.33

(57) ABSTRACT

Provided herein are molecular assays, including oligonucleotides and other reagents, for the detection and analysis of PRAME (Preferentially Expressed Antigen in Melanoma). The assays find use, for example, as diagnostic and prognostic applications, including use in assessing therapeutic courses of action.

The present application claims priority to U.S. Provisional Patent Application Ser. No. 61/695,180, filed Aug. 30, 2012, the disclosure of which is herein incorporated by reference in its entirety.

### FIELD OF THE INVENTION

Provided herein are molecular assays, including oligonucleotides and other reagents, for the detection and analysis of PRAME (Preferentially Expressed Antigen in Melanoma). The assays find use, for example, as diagnostic and prognostic applications, including use in assessing therapeutic courses of action.

### BACKGROUND

The PRAME (Preferentially Expressed Antigen in Melanoma) gene is expressed at low or undetectable levels in most normal tissues. However, PRAME is expressed at high levels in a large fraction of cancers, making it a potentially useful target for anti-cancer therapy and for minimal residual disease monitoring. PRAME expression has also been reported to correlate with prognosis and clinical survival in some cancer types. Thus, assessment of PRAME expression may serve as an important diagnostic and/or prognostic tool in oncology.

### SUMMARY OF THE INVENTION

The present invention provides molecular assays, including oligonucleotides and other reagents, for the detection and analysis of PRAME (Preferentially Expressed Antigen in Melanoma). The assays find use, for example, as diagnostic and prognostic applications, including use in assessing therapeutic courses of action.

In some embodiments, the present invention provides methods of detecting the presence or amount of PRAME (Preferentially Expressed Antigen in Melanoma) mRNA in a sample comprising: a) contacting a sample suspected of containing PRAME mRNA with a first primer and a second primer under conditions such that a first amplification product is generated, wherein the first primer hybridizes to Exon 3 of the PRAME mRNA, and wherein the second primer hybridizes to Exon 4 of the PRAME mRNA; and b) detecting the first amplification product, thereby determining the presence and/or amount of the PRAME in the sample.

In certain embodiments, the contacting does not generate detectable amplicons from genomic PRAME. In further embodiments, the contacting does not generate detectable amplicons from any PRAME-like mRNAs or genes (e.g., from the twenty-two known PRAME-like genes or encoded mRNA).

In further embodiments, the first primer hybridizes within the final 100 bases at the 3′ end of Exon 3 of the PRAME mRNA. In other embodiments, the first primer hybridizes within the final 50 (or final 40 . . . 35 . . . 30 . . . 25 . . . or 20) bases at the 3′ end of Exon 3 of the PRAME mRNA. In certain embodiments, the first primer comprises, or consists of, at least 70% sequence identity with a sequence selected from the group consisting of SEQ ID NOs:3-6 and 36 (e.g., at least 70% . . . 80% . . . 90% . . . 95% . . . 98% . . . or 99% sequence identity).

In other embodiments, the second primer hybridizes within the first 100 bases at the 5′ end of Exon 4 of the PRAME mRNA. In particular embodiments, the second primer hybridizes within the first 50 (or first 40 . . . 35 . . . 30 . . . 25 . . . or 20) bases at the 5′ end of Exon 4 of the PRAME mRNA. In further embodiments, the second primer comprises at least 70% sequence identity with a sequence selected from the group consisting of SEQ ID NOs:7-10 and 35 (e.g., at least 70% . . . 80% . . . 90% . . . 95% . . . 98% . . . or 99% sequence identity).

In some embodiments, the sample is from a formalin fixed paraffin embedded sample. In other embodiments, the sample comprises a tumor sample or blood sample. In particular embodiments, the tumor sample is selected from the group consisting of: a melanoma tumor sample, a NSCLC sample, an ovarian tumor sample, a bladder, a head and neck tumor sample, a breast cancer sample, and a myeloma sample. In certain embodiments, the blood sample is suspected of containing leukemia cells.

In further embodiments, the methods further comprise contacting the sample with a first probe that hybridizes to the first amplicon (e.g., the first probe hybridizes across the Exon 3-Exon 4 junction present in the amplicon of the PRAME mRNA). In some embodiments, the first probe comprises at least 70% sequence identity with a sequence selected from the group consisting of SEQ ID NOs:11-16 (e.g., at least 70% . . . 80% . . . 90% . . . 95% . . . 98% . . . or 99% sequence identity).

In certain embodiments, the first and second primers are a primer pair selected from the group consisting of: SEQ ID NOs:3 and 7; SEQ ID NOs:4 and 8; SEQ ID NOs: 5 and 9; and SEQ ID NOs:6 and 10. In other embodiments, the first and second primers are each about 10 to 40 nucleotides in length.

In certain embodiments, the detection assays described herein are used to identify cancer patients in need of cancer treatment. For example, in some embodiments, the patients are identified as in need of PRAME or MAGEA3 immunotherapy. In some embodiments, the primers and/or probes described herein are used in a method, other than standard polymerase chain reaction, such as sequencing, LATE-PCR, whole genome amplification, etc. In certain embodiments, the amplicons generated with the methods described herein are described via detectable labels on the probes (e.g., fluorescent labels, with or without quenchers). In certain embodiments, the amplicons are detected by mass spectrometry.

In particular embodiments, the methods further comprise contacting the sample with a third primer and fourth primer under conditions such that a second amplification product is generated, wherein the second amplification product comprises an amplified portion of an internal control mRNA sequence (e.g., housekeeping mRNA sequence). In other embodiments, the housekeeping mRNA sequence is beta-actin mRNA.

In some embodiments, the sample is further suspected of containing beta-actin mRNA, and wherein the method further comprises: c) contacting the sample with a third primer and a fourth primer under conditions such that a second amplification product is generated, wherein the third primer hybridizes to Exon 4 of the beta-actin mRNA, and wherein the fourth primer hybridizes to Exon 5 of the beta-actin mRNA, d) detecting the second amplification product.

In certain embodiments, the third primer hybridizes within the final 100 bases at the 3′ end of Exon 4 of the beta-actin mRNA. In other embodiments, the third primer hybridizes within the final 50 (or final 40 . . . 35 . . . 30 . . . 25 . . . or 20) bases at the 3′ end of Exon 4 of the beta-actin mRNA. In some embodiments, the third primer comprises at least 70% sequence identity with a sequence selected from the group consisting of SEQ ID NOs:17-23 and 32 (e.g., at least 70% . . . 80% . . . 90% . . . 95% . . . 98% . . . or 99% sequence identity).

In further embodiments, the fourth primer hybridizes within the first 100 bases at the 5′ end of Exon 5 of the beta-actin mRNA. In other embodiments, the fourth primer hybridizes within the first 50 (or first 40 . . . 35 . . . 30 . . . 25 . . . or 20) bases at the 5′ end of Exon 5 of the beta-actin mRNA. In other embodiments, the fourth primer comprises at least 70% sequence identity with a sequence selected from the group consisting of SEQ ID NOs:24-27 and 33 (e.g., at least 70% . . . 80% . . . 90% . . . 95% . . . 98% . . . or 99% sequence identity).

In some embodiments, the methods further comprises contacting the sample with a second probe, wherein the second probe hybridizes across the Exon 4-Exon 5 beta-actin mRNA junction present in the second amplification product. In certain embodiments, the second probe comprises at least 70% sequence identity with a sequence selected from the group consisting of SEQ ID NOs:28-31 (e.g., at least 70% . . . 80% . . . 90% . . . 95% . . . 98% . . . or 99% sequence identity).

In particular embodiments, the present invention provides kits and composition comprising a first isolated probe or primer about 15-50 bases in length which comprises or consists of a sequence selected from the group consisting of SEQ ID NOs: 3-36 or a sequence with at least 70% sequence identity with SEQ ID NOs:3-36 (e.g., at least 70% . . . 80% . . . 90% . . . 95% . . . 98% . . . or 99% sequence identity). In some embodiments, the kits and compositions further comprise a second isolated probe or primer 15-50 bases in length which comprises or consists of a sequence selected from the group consisting of SEQ ID NOs: 3-36 (or a sequence with at least 70% sequence identity with SEQ ID NOs:3-33) and which has a different sequence than the first isolated probe or primer. In further embodiments, the kits and compositions further comprise a third isolated probe or primer 15-50 bases in length which comprises or consists of a sequence selected from the group consisting of SEQ ID NOs: 3-36 (or a sequence with at least 70% sequence identity with SEQ ID NOs:3-36) and which has a different sequence than the first isolated probe or primer and the second isolated probe or primer. In further embodiments, the kits and compositions further comprise a fourth (and fifth, sixth, seventh, etc.) isolated probe or primer 15-50 bases in length which comprises or consists of a sequence selected from the group consisting of SEQ ID NOs: 3-36 (or a sequence with at least 70% sequence identity with SEQ ID NOs:3-36) and which has a different sequence than said first, second, and third isolated probes or primers.

In some embodiments, the present invention provides kits and compositions comprising at least one purified oligonucleotide primer pair that comprises forward and reverse primers that are about 15 to 50 nucleobases in length, and wherein the forward primer comprises at least 70% sequence identity with a sequence selected from the group consisting of SEQ ID NOS: 3-6 and 36, and the reverse primer comprises at least 70% sequence identity with a sequence selected from the group consisting of SEQ ID NOS: 7-10 and 35 (e.g., at least 70% . . . 80% . . . 90% . . . 95% . . . 98% . . . or 99% sequence identity). In further embodiments, the kits and compositions further comprise a probe about 15-70 bases in length that comprises at least 70% sequence identity with a sequence selected from the group consisting of SEQ ID NOs: 11-16 (e.g., at least 70% . . . 80% . . . 90% . . . 95% . . . 98% . . . or 99% sequence identity).

In some embodiments, the present invention provides kits and compositions comprising at least one purified oligonucleotide primer pair that comprises forward and reverse primers that are about 15 to 50 nucleobases in length, and wherein the forward primer comprises at least 70% sequence identity with a sequence selected from the group consisting of SEQ ID NOS: 17-23 and 32 (e.g., at least 70% . . . 80% . . . 90% . . . 95% . . . 98% . . . or 99% sequence identity), and the reverse primer comprises at least 70% sequence identity with a sequence selected from the group consisting of SEQ ID NOS: 24-27 and 33 (e.g., at least 70% . . . 80% . . . 90% . . . 95% . . . 98% . . . or 99% sequence identity). In further embodiments, the kits and compositions further comprise a probe 15-70 bases in length that comprises at least 70% sequence identity with a sequence selected from the group consisting of SEQ ID NOs: 28-31 (e.g., at least 70% . . . 80% . . . 90% . . . 95% . . . 98% . . . or 99% sequence identity).

### DESCRIPTION OF THE FIGURE

FIG. 1A shows a partial sequence of the Exon 3/Exon 4 boundary found in human PRAME mRNA (SEQ ID NO:1) and arrows showing where a forward primer, a reverse primer, and probe hybridize to detect PRAME mRNA in a sample. FIG. 1B shows a partial sequence of the Exon 4/Exon 5 boundary found in human beta-Actin mRNA (SEQ ID NO:2) and arrows showing where a forward primer, a reverse primer, and probe hybridize to detect beta-actin mRNA in a sample.

### DEFINITIONS

As used herein, the term “about” means encompassing plus or minus 10%. For example, about 100 nucleotides refers to a range encompassing between 110 and 90 nucleotides.

As used herein, the term “amplicon” or “amplification product” refers to a nucleic acid generated using the primer pairs described herein. The amplicon is typically double stranded DNA; however, it may be RNA and/or DNA:RNA.

Amplicons typically comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One of ordinary skill in the art will further appreciate that the above range is not an absolute limit to the length of an amplicon, but instead represents a preferred length range. Amplicon lengths falling outside of this range are also included herein.

The term “amplifying” or “amplification” in the context of nucleic acids refers to the production of multiple copies of a polynucleotide (e.g., mRNA sequence), or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. The present invention includes sequences complementary to all of the primer and probe sequences described herein.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length sequence or fragment thereof are retained. As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers and probes of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (Tₘ) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods described herein. Samples can include, for example, a tumor sample, evidence from a crime scene, blood, blood stains, semen, semen stains, bone, teeth, hair saliva, urine, feces, fingernails, muscle tissue, cigarettes, stamps, envelopes, dandruff, fingerprints, personal items, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved binding sequence of a nucleic acid (e.g., PRAME mRNA sequence). Similarly, the primer pairs provided herein may comprise between about 70%-100%, or between about 80-100%, or between about 90-100%, or between about 95-100% identity, or between about 99-100% sequence identity with the primer pairs disclosed in Tables 1 and 2. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less then 70% sequence homology with a corresponding member of any of the primer pairs of Tables 1 and 2 if the primer pair has the capability of producing an amplification product corresponding to the desired amplicon.

### DETAILED DESCRIPTION

The present invention provides molecular assays, including oligonucleotides and other reagents, for the detection and analysis of PRAME (Preferentially Expressed Antigen in Melanoma). The assays find use, for example, as diagnostic and prognostic applications, including use in assessing therapeutic courses of action. In particular embodiments, the present invention provides PRAME primers and probes, as well as beta-actin primers and probes. In certain embodiments, the primers and probes are useful for detecting PRAME mRNA, and particularly the Exon 3/4 boundary of PRAME mRNA. In some embodiments, the primers and probes are useful for detecting beta-actin mRNA (e.g., as an internal control to allow quantitation of PRAME mRNA), and particularly the Exon 4/5 boundary of beta-actin.

In certain embodiments, the present invention provides a diagnostic method and assay format that utilizes PRAME primer/probes to achieve highly specific and sensitive detection of PRAME mRNA expression using real-time RT-PCR technology. The invention also provides primer/probe sets for detection of mRNA from the endogenous housekeeping gene beta-Actin, which can be used, for example: 1) to facilitate the quantitation of PRAME relative expression, and/or 2) as a sample validity control for cell adequacy, sample extraction, and amplification efficiency.

In certain embodiments, the present invention provides amplification and detection of RNA expression from the PRAME gene. In certain embodiments, two oligonucleotide primers (Prp and Pfp) and one hybridization probe (Pprobe) may be used. For example, reverse primer Prp can anneal to a sequence within PRAME exon 4 and directs reverse transcription of PRAME RNA. Forward Primer Pfp can anneal to a sequence within PRAME exon 3, and in combination with reverse primer Prp, can direct the amplification of the PRAME cDNA reverse transcribed in the step above. Since PRAME Prp and Pfp are located in different exons, the resulting amplicon contains an RNA-specific sequence at the PRAME exon 3/exon 4 boundary that is not present in genomic DNA (gDNA). In certain embodiments, the detection principle for this invention utilizes a labeled oligonucleotide probe designed to specifically hybridize to the PRAME exon 3/exon 4 junction sequence contained within the amplicon, thereby ensuring that real-time PCR signal generated by the probe is specific for PRAME RNA/cDNA (and not genomic DNA). Exemplary PRAME primers and probes are shown in Table 1 below.

TABLE 1

| Designation | Description | Sequence (5′ to 3′) |
|---|---|---|
| Prp | PRAME reverse primer | TCCACACACTCATGCTGATGTATCG (SEQ ID NO: 7) |
| Pfp | PRAME forward primer | CCTAAGTCGCTTCAAAATGGAACG (SEQ ID NO: 3) |
| Pprobe | PRAME probe | Dye AAGGCGTTTGTGGGGTTCCATTCAGA Quencher (SEQ ID: 11) |
| Prp_1 | PRAME reverse primer | TCATGCTGATGTATCGGCTCTGAA (SEQ ID NO: 9) |
| Prp_2 | PRAME reverse primer | ACACTCATGCTGATGTATCGGCTCT (SEQ ID NO: 10) |
| Pfp_1 | PRAME forward primer | GTCGCTTCAAAATGGAACGAAGG (SEQ ID NO: 5) |
| Pfp_2 | PRAME forward primer | CAGCCTAAGTCGCTTCAAAATGGA (SEQ ID NO: 6) |
| Pprobe_1 | PRAME probe | Dye ACGAAGGCGTTTGTGGGGTTCCATTC Quencher (SEQ ID: 14) |
| Pprobe_2 | PRAME probe | Dye CGTTTGTGGGGTTC Quencher (SEQ ID NO: 15) |
| Pprobe_3 | PRAME probe | Dye TTTGTGGGGTTCC Quencher (SEQ ID NO: 16) |
| Pprobe_4 | PRAME probe | CY5 TTGTGGGGTTCC BHQ (SEQ ID NO: 13) |

In Table 1, the probe sequence from the 3′ end of PRAME exon 3 is represented in italics. Probe sequence from the 5′ end of PRAME exon 4 is represented in Bold. The complementary sequence could also be used as a probe if desired. Also, the label at the opposite end from the dye can be any moiety that can effectively quench the fluorescent dye and achieve effective association of the probe to its target sequences (e.g., black hole quencher can be used). In the above table, the dye is labeled at the 5′ end of the probe and the quencher at the 3′ end, the design also applies to the use of a dye labeled at the 3′ end and a quencher at the 5′ end. In certain embodiments, the primers and probe can be modified to include a variety of binding enhancers such as, for example, mgb or pdU/pdC.

In certain embodiments, the present invention also provides amplification and detection of RNA from an endogenous gene for use as an internal control. For example, two oligonucleotide primers (bArp and bAfp) and one hybridization probe (bAprobe) may be used to detect the house-keeping gene beta-Actin. For example, the reverse primer bArp anneals to a sequence within beta-Actin exon 5 and directs reverse transcription of beta-Actin RNA. The forward Primer bAfp anneals to a sequence within beta-Actin exon 4, and in combination with Reverse Primer bArp, directs the amplification of the beta-Actin cDNA reverse transcribed in the step above. Since bArp and bAfp are located in different exons, the resulting amplicon contains an RNA-specific sequence at the beta-Actin exon 4/exon 5 boundary that is not present in gDNA. In certain embodiments, the present invention utilizes a labeled oligonucleotide probe (bAprobe) designed to specifically hybridize to the beta-Actin exon 4/exon 5 junction sequence contained within the amplicon, thereby ensuring that real-time PCR signal generated by the probe is specific for beta-Actin RNA/cDNA (and not gDNA). In certain embodiments, the beta-Actin primers and probe can be included with the PRAME primer and probe mix for amplification/detection of PRAME RNA and beta-Actin RNA simultaneously. Note that in such a configuration, bAprobe is generally labeled with a unique dye to allow differentiation of beta-Actin signal from PRAME signal. Exemplary beta-actin probes and primers are provided in Table 2.

TABLE 2

| Designation | Description | Sequence (5′ to 3′) |
|---|---|---|
| bArp | beta-Actin reverse primer | TGGAGTTGAAGGTAGTTTCGTGGATG (SEQ ID NO: 24) |
| bAfp | beta-Actin forward primer | TGCCCTGAGGCACTCTTCCAG (SEQ ID NO: 17) |
| bAprobe | beta-Actin probe | Dye CCTTCCTTCCTGGGCATGGAGTCCT Quencher (SEQ ID: 28) |
| bArp_1 | beta-Actin reverse primer | TGAAGGTAGTTTCGTGGATGCCA (SEQ ID NO: 33) |
| bAfp_1 | beta-Actin forward primer | GCCCTGAGGCACTCTTCCAGC (SEQ ID NO: 32) |

In Table 2, the probe sequence from the 3′ end of beta-Actin exon 4 is represented in italics. The probe sequence from the 5′ end of beta-Actin exon 5 is represented in Bold. The complementary sequence could also be used as a probe, if desired. Also, the label at the opposite end from the dye can be any moiety that can effectively quench the fluorescent dye and achieve effective association of the probe to its target sequences. In the above table, the dye is labeled at the 5′ end of the probe and the quencher at the 3′ end, the design also applies to the use of a dye labeled at the 3′ end and a quencher at the 5′ end. In certain embodiments, the primers and probe can be modified to include a variety of binding enhancers such as mgb or pdU/pdC.

Additional PRAME and beta-actin primers and probes that may be used in the methods, kits, and compositions of the present invention are shown in Table 3 below.

TABLE 3

| | | SEQ ID |
|---|---|---|
| Primer/Probe | SEQUENCE | NO. |
| PRAMEi3_fwd−44to−24 | AGGCCAGCCTAAGTCGCTTCA | 4 |
| PRAME13_rev+34to+10 | CACTCATGCTGATGTATCGGCTCTG | 8 |
| PRAMEi3fwd−16to+10pCY5 | CY5 - ACGAAGGCGTTTGTGGGGTTCCATTC - BHQ2 | 12 |
| AM PR 3 | Quasar - pdCGpdUTpdUGpdUGGGGpdUpdUpdCpdCApdUpdU - BHQ2 | 34 |
| PRMrev_1 | CACACTCATGCTGATGTATCGG | 35 |
| PRMfwd_1 | AAGTCGCTTCAAAATGGAACG | 36 |
| bAct fwd_a | TGCCCTGAGGCACTCTTCCA | 18 |
| bAct probe_a | CCTTCCTTCCTGGGCATGGAGTC | 29 |
| bAct fwd_b | TGCCCTGAGGCACTCTTCC | 19 |
| bAct fwd_c | GCCCTGAGGCACTCTTCCAG | 20 |
| bAct probe_b | CTTCCTTCCTGGGCATGGAGTC | 30 |
| bAct fwd_d | CCCTGAGGCACTCTTCCAG | 21 |
| bAct rev_a | GGAGTTGAAGGTAGTTTCGTGGA | 27 |
| bActI5_−34to−14 | TGCCCTGAGGCACTCTTCCAG | 17 |
| bActI5+42to+17 | TGGAGTTGAAGGTAGTTTCGTGGATG | 24 |
| bActI5−13to+12pVIC | VIC - CCTTCCTTCCTGGGCATGGAGTCCT - BHQ | 31 |
| bActI4_−34to−18 | TGCCCTGAGGCACTCTTCC | 22 |
| bActI4_+40to+17 | GAGTTGAAGGTAGTTTCGTGGATG | 25 |
| bActI4_−32to−14 | CCCTGAGGCACTCTTCCAG | 23 |
| bActI4_+42to+20 | TGGAGTTGAAGGTAGTTTCGTGG | 26 |

The primers and probes may be used in diagnostic methods, such as the following exemplary methods. First, a cocktail is formed containing the primers and probes as well as other components essential for nucleic acid amplification, such as dNTP mix, buffer, polymerase enzyme and divalent ion as activation reagent. A reaction mixture is formed with the cocktail and the purified RNA potentially containing PRAME transcripts. The reaction mixture is then subjected to conditions for reverse transcription of the target PRAME and endogenous control gene transcripts using reverse primers. Then, one amplifies the copy number of the resulting cDNA sequences with the forward/reverse primer sets and detects the presence of amplified PRAME and/or control gene target sequences with probes. The one can determine the PRAME transcript levels relative to the beta-Actin control gene transcript levels (e.g., for relative quantitation).

In certain embodiments, the methods employ a polymerase enzyme capable of catalyzing both the reverse transcription of RNA sequences and the amplification of DNA copy number. In such embodiments, the reverse transcription and amplification steps can, for example, be carried out in a single-run, closed-tube format by an instrument capable of concurrent thermal cycling and signal detection.

In certain embodiments, since the methods are capable of accommodating multiple primer and probe sets within the cocktail, it allows detection and differentiation of RNA from PRAME and the endogenous internal gene to be carried out in a single reaction. In some embodiments, the PRAME and beta-Actin primer/probe sets are run in multiplex within the same reaction. In such embodiments, the PRAME and beta-Actin probes are generally labeled with different dyes to allow differentiation of PRAME signal from beta-Actin signal. PRAME mRNA levels in the sample are then quantitated relative to endogenous beta-Actin mRNA levels. Detection of beta-Actin mRNA also serves as a within-well sample validity control for cell adequacy, sample extraction, and amplification efficiency.

In particular embodiments, the PRAME and beta-Actin primer/probe sets are run in singleplex within separate reactions (e.g., where each reaction contains an aliquot of the same RNA sample). In this configuration, PRAME mRNA levels in the sample can be quantitated relative to endogenous beta-Actin mRNA levels using between-well results from the independent PRAME and beta-Actin reactions. The PRAME primer/probe set can also be used independently for qualitative detection of PRAME transcript levels in an RNA sample. Alternately, the PRAME primer/probe set can be used in combination with a different endogenous control primer/probe set (beta-Actin or non-beta-Actin) for relative quantification of PRAME transcripts.

The beta-Actin primer/probe sets can also be used in combination with other primer/probe sets (e.g., PRAME or non-PRAME) to serve, for example, as: 1) an endogenous internal control for relative quantitation of mRNA levels from targeted genes and/or 2) a sample validity control for cell adequacy, sample extraction, and amplification efficiency.

In certain embodiments, the present invention provides primers and probes designs capable of achieving highly sensitive and specific detection of PRAME and beta-Actin mRNA. In combination, the PRAME and beta-Actin RT-PCR signals can be used to measure relative expression of PRAME mRNA. These primers/probes may be used, for example, in an Abbott RealTime PRAME assay that detects relative PRAME expression in non-small cell lung cancer (NSCLC). The primers/probes can also be applied to a variety of additional cancer types for which detection of PRAME expression may be of diagnostic or prognostic value.

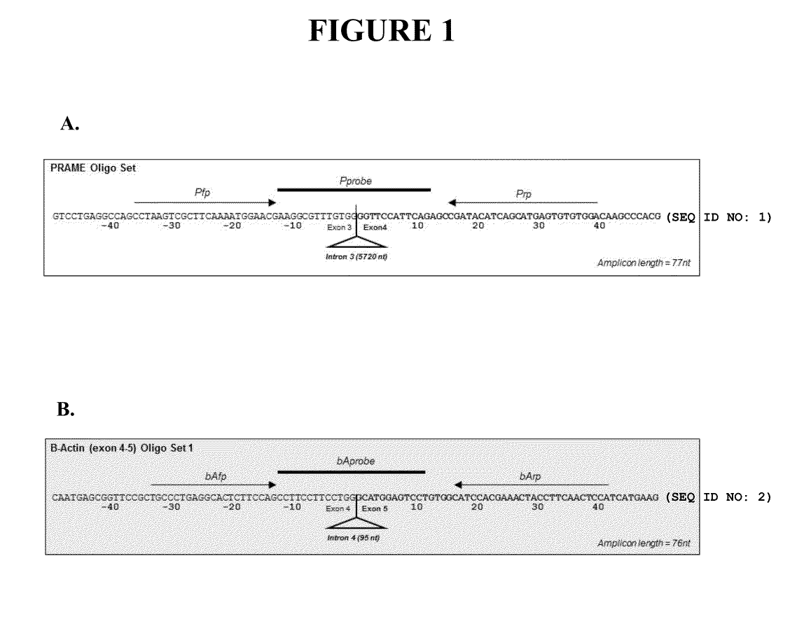

In certain embodiments, the PRAME and beta-Actin primer described in this invention sets are designed to generate short PCR amplicons that are approximately 75 nt in length (see, e.g., FIG. 1). These short amplicons are conducive for PCR amplification from FFPE-derived RNA, which is typically highly fragmented due to the chemical fixation process used to prepare FFPE tissue blocks. In other embodiments, the present invention can also be effectively used with RNA from non-FFPE tissue sources including, but not limited to, frozen tumor tissues, fine needle biopsy aspirates, whole blood, and bone marrow aspirates.

In particular embodiments, the primers and probes of the present invention are designed to specifically detect mRNA expression from PRAME (and beta-Actin) and do not cross-react with gDNA sequences. In contrast, all tissues (normal and cancer) are expected to contain PRAME (and beta-Actin) gDNA sequences. Therefore, detection of PCR signals emanating from residual gDNA present in RNA samples has the potential to cause undesired or inaccurate assay results (e.g., false-positives).

The PRAME primers and probe featured in this invention are also generally designed to specifically amplify and detect mRNA expressed from the PRAME gene and not from the 22 closely related PRAME-like gene family members that are known to be present in the human genome. This design feature ensures that transcripts detected by the invention are specific to PRAME (vs. PRAME-like).

Exemplary uses of the present invention include, but are not limited to: target amplification, detection and/or relative quantification of RNA transcribed from the PRAME gene (and from an endogenous control gene) either as a complete cocktail or in a subset of primer/probe combinations. Described below is an exemplary set of PCR formulations and cycling conditions. For example, the PCR reaction may be formed with 25 ul of target and 25 ul of PCR reagent mixture as detailed in Table 4 below:

TABLE 4

| Component | Reaction Concentration | Unit of Measure |
|---|---|---|
| Prp | 0.300 | μM |
| Pfp | 0.100 | μM |
| Pprobe | 0.300 | μM |
| bArp | 0.300 | μM |
| bAfp | 0.100 | μM |
| bAprobe | 0.300 | μM |
| HIV EZ buffer | 1.0 | X |
| dNTPs | 0.325 | mM |
| ROX reference dye | 0.015 | uM |
| Aptamer | 0.200 | uM |
| rTth Polymerase | 10.0 | Units |
| MnCl2 | 3.000 | mM |

Exemplary PCR cycling conditions are shown in Table 5 below:

TABLE 5

| Cycles | Parameters | Description |
|---|---|---|
| 1 | 62° C./30 min | Reverse Transcription |
| 4 | 92° C./30 sec, 60° C./30 sec* | DNA Amplification |
| 50 | 92° C./30 sec, 62° C./30 sec*, | DNA Amplification |
| | 58° C./40 sec | (DNA Amplification |
| | | and Fluorescence Reads) |

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising PRAME mRNA, a first primer or probe that hybridizes to Exon 3 of said PRAME mRNA, and a second primer or probe that hybridizes to Exon 4 of said PRAME mRNA.

2. The composition of claim 1, further comprising an amplification product generated by the amplification of said PRAME mRNA with said first primer and said second primer.

3. The composition of claim 2, further comprising a probe that is complementary to said amplification product.

4. The composition of claim 3, wherein said probe comprises a label.

5. The composition of claim 4, wherein said label is a fluorescent label.

6. The composition of claim 1, wherein said first primer or probe is 15-50 bases and length and comprises or consists of a sequence selected from the group consisting of SEQ ID NOs: 3-36.

7. The composition of claim 6, wherein said second primer or probe is 15-50 bases in length and comprises or consists of a sequence selected from the group consisting of SEQ ID NOs: 3-36 and which has a different sequence than said first primer or probe.

8. The composition of claim 7, further comprising a third isolated primer or probe 15-50 bases in length which comprises or consists of a sequence selected from the group consisting of SEQ ID NOs: 3-36 and which has a different sequence than said first isolated primer or probe and said second isolated primer or probe.

9. The composition of claim 2, wherein said composition does not contain detectable amplicons generated from genomic PRAME.

10. The composition of claim 2, wherein said composition does not contain detectable amplicons generated from any PRAME-like mRNAs or genes.

11. The composition of claim 1, wherein said first primer hybridizes within the final 100 bases at the 3′ end of Exon 3 of said PRAME mRNA.

12. The composition of claim 1, wherein said first primer hybridizes within the final 50 bases at the 3′ end of Exon 3 of said PRAME mRNA.

13. The composition of claim 1, wherein said first primer comprises at least 70% sequence identity with a sequence selected from the group consisting of SEQ ID NOs:3-6 and 36.

14. The composition of claim 1, wherein said second primer hybridizes within the first 100 bases at the 5′ end of Exon 4 of said PRAME mRNA.

15. The composition of claim 1, wherein said second primer hybridizes within the first 50 bases at the 5′ end of Exon 4 of said PRAME mRNA.

16. The composition of claim 1, wherein said second primer comprises at least 70% sequence identity with a sequence selected from the group consisting of SEQ ID NOs:7-10 and 35.

17. The composition of claim 1, further comprising a paraffin embedded sample.

18. The composition of claim 17, wherein said sample comprises a tumor sample.

19. The composition of claim 18, wherein said tumor sample is selected from the group consisting of: a melanoma tumor sample, a NSCLC sample, an ovarian tumor sample, a bladder, a head and neck tumor sample, a breast cancer sample, and a myeloma sample.

20. The composition of claim 1, comprising said first primer and said second primer and further comprising a probe wherein said probe hybridizes across the Exon 3-Exon 4 PRAME mRNA junction present in an amplicon generated by said first primer and said second primer with said PRAME mRNA.

21. The composition of claim 20, wherein said probe comprises at least 70% sequence identity with a sequence selected from the group consisting of SEQ ID NOs:11-16.

22. The composition of claim 1, wherein said first and second primers are a primer pair selected from the group consisting of: SEQ ID NOs:3 and 7; SEQ ID NOs:4 and 8; SEQ ID NOs: 5 and 9; and SEQ ID NOs:6 and 10.

23. The composition of claim 1, further comprising a third primer and fourth primer that are complimentary to a housekeeping mRNA.

24. The composition of claim 23, further comprising said housekeeping mRNA.

25. The composition of claim 24, wherein said housekeeping mRNA is beta-actin mRNA.

26. The composition of claim 1, wherein said composition comprises a reaction mixture.

27. A kit comprising at least one purified oligonucleotide primer pair that comprises forward and reverse primers that are about 15 to 50 nucleobases in length, and wherein said forward primer comprises at least 70% sequence identity with a sequence selected from the group consisting of SEQ ID NOS: 3-6 and 36, and said reverse primer comprises at least 70% sequence identity with a sequence selected from the group consisting of SEQ ID NOS: 7-10 and 35.

28. The kit or composition of claim 27, further comprising a probe 15-70 bases in length that comprises at least 70% sequence identity with a sequence selected from the group consisting of SEQ ID NOs: 11-16.

29. A method of detecting the presence or amount of PRAME (Preferentially Expressed Antigen in Melanoma) mRNA in a sample comprising:
a) contacting a sample suspected of containing PRAME mRNA with a first primer and a second primer under conditions such that a first amplification product is generated, wherein said first primer hybridizes to Exon 3 of said PRAME mRNA, and wherein said second primer hybridizes to Exon 4 of said PRAME mRNA; and
b) detecting said first amplification product, thereby determining the presence and/or amount of said PRAME in said sample.

30. The method of claim 29, wherein said contacting does not generate detectable amplicons from genomic PRAME.

31. The method of claim 29, wherein said contacting does not generate detectable amplicons from any PRAME-like mRNAs or genes.
